(19) United States
(12) Patent Application Publication
(10) Pub. No.: US 2021/0275393 A1
(43) Pub. Date: Sep. 9, 2021

(54) REST FOR A MATTRESS SUPPORT SYSTEM AND METHOD FOR SENSORY TRAINING OF A PERSON

(71) Applicant: Thomas GmbH + Co. Technik + Innovation KG, Bremervörde (DE)

(72) Inventor: Klaus Jansen, Buxtehude (DE)

(73) Assignee: Thomas GmbH + Co. Technik + Innovation KG, Bremervörde (DE)

(21) Appl. No.: 16/336,700

(22) PCT Filed: Oct. 6, 2017
(86) PCT No.: PCT/EP2017/001185

§ 371 Date: Mar. 26, 2019

(30) Foreign Application Priority Data
Oct. 14, 2016 (DE) 102016012244.7

(51) Int. Cl. A61H 39/00; A61H 39/06; A61N 5/06

(52) U.S. Cl. CPC A61H 39/007; A61H 39/06; A61H 39/00; A61N 5/0619; A61H 2201/0207; A61H 2039/005; A61H 2201/5097; A61H 2201/0146; A61N 2005/0659; A61N 2005/0663; A61N 2005/0667; A61H 2201/0214

(57) ABSTRACT

A rest for a mattress support system and which serves for sensory training of a person, and a method for sensory training of a person. This rest has a multiplicity of actuatable micro-stimulating elements for producing sensory stimuli. These elements are assigned to the rest. Using a control device, the person can actuate the micro-stimulating elements during the training. Moreover, this control device serves to generate a feedback signal in response to the sensory stimuli.

### CROSS REFERENCE TO RELATED APPLICATIONS

This application is the US National Phase of and claims the benefit of and priority on International Application No. PCT/EP2017/001185 having a filing date of 6 Oct. 2017, which claims priority on and the benefit of German Patent Application No. 10 2016 012 244.7 having a filing date of 14 Oct. 2016.

### BACKGROUND OF THE INVENTION

### Technical Field

The invention relates to a rest for a mattress support system, in particular for a mattress of an item of furniture for sleeping on, sitting on or lying on, for sensory training of a person, with a multiplicity of micro-stimulating elements which can be actuated individually or in groups and trigger sensory stimuli and which are arranged on a surface of the rest or are integrated in the rest, and with a control device having means for active or passive actuation of the micro-stimulating elements, individually or in groups, by the person during the training and for generating a feedback signal in response to the sensory stimuli. The invention further relates to a method for sensory training of a person using a rest for a mattress support system, in particular for a mattress of an item of furniture for sleeping on, sitting on or lying on, wherein the rest has a multiplicity of micro-stimulating elements which can be actuated individually or in groups and trigger sensory stimuli and which can be actively or passively controlled by the person, either individually or in groups, via a control device, wherein the passive control involves the use of a feedback signal which is generated by the control device on the basis of the reaction of the person to the stimuli.

### Prior Art

In known mattress support systems, in particular for a mattress of an item of furniture for sleeping on, sitting on or lying on, for treatment of conditions such as dementia, Parkinson's, decubitus ulcers or coma, massaging means are used to trigger certain stimuli in a person who is lying on the mattress support. For this purpose, a massage function in the mattress support is generally initiated, which exerts a physical force on the person in a random fashion, i.e. without selecting specific body regions.

However, it is also known for individual massage elements or groups of these elements to be actuated in order to subject only specific regions of the body to a periodic and mechanical force. Depending on the condition that has been diagnosed, different regions of the body can then be treated with very different mechanical stimulations. The nature and intensity of this treatment is in most cases determined by the person who is carrying out the treatment. This treatment often follows empirical values that have already been applied to persons requiring treatment for a similar clinical picture.

A particular disadvantage of this method has been that the person who is to be treated has no possibility of controlling the treatment himself. As a result, the known methods and techniques have proven extremely inefficient.

### BRIEF SUMMARY OF THE INVENTION

The object of the invention is therefore to make available a rest for a mattress support system and also a method for sensory training, by which particularly efficient treatment of a person is made possible.

This object is achieved by a rest for a mattress support system, in particular for a mattress of an item of furniture for sleeping on, sitting on or lying on, for sensory training of a person, with a multiplicity of micro-stimulating elements which can be actuated individually or in groups and trigger sensory stimuli and which are arranged on a surface of the rest or are integrated in the rest, and with a control device having means for active or passive actuation of the micro-stimulating elements, individually or in groups, by the person during the training and for generating a feedback signal in response to the sensory stimuli. Accordingly, the invention proposes a rest for a mattress support system, in particular for a mattress of an item of furniture for sleeping on, sitting on or lying on, for sensory training of a person. This rest has a multiplicity of micro-stimulating elements which can be actuated individually or in groups and trigger sensory stimuli. These elements are arranged on a surface of the rest or are directly integrated in the rest. According to the invention, the rest moreover has a control device with means allowing active or passive actuation of the micro-stimulating elements, either individually or in groups, by the person during the course of training. This control device moreover serves to generate a feedback signal in response to the sensory stimuli. By virtue of this rest, in particular by virtue of this control device, the person is therefore not only able to arrange or control the training himself; rather, the control device allows the person to generate a feedback signal in response to the sensory stimuli. With this feedback signal, it is possible in particular to establish whether the sensory stimuli produced are perceptible to the person. Depending on this feedback signal, the micro-stimulating elements can be actuated by the person himself, or by the control device, for the further training. However, provision is also made according to the invention that the micro-stimulating elements are actuated passively. This proves advantageous in particular for the treatment of comatose patients who are unable to actively operate the control device.

Preferably, provision can also be made according to the present invention that the micro-stimulating elements are arranged on and/or in the rest at a spacing equal to, in particular below, the discrimination threshold of the human body for the perception of stimuli, this spacing being variable for different body regions of the person. In particular, in order to improve awareness of the body and/or to improve the perception of the body image of the person, the discrimination threshold is tested between two locally offset stimuli. It is specifically determined how precisely the person is able to define a location of a stimulus in relation to his own body. In order not just to be able to determine but also improve the value of this discrimination threshold of the person, it is essential that the spacing of the micro-stimulating elements is very small. It is considered particularly advantageous if the spacings are able to be varied. Thus, rests can be configured individually for different persons with different physiognomies and for different regions of the body. For example, it is conceivable that the elements can be placed at different positions on or in or under the rest by way of hook-and-loop fasteners, buttons, magnets or the like. However, it is also conceivable that the micro-stimulating elements are connected to the rest in a fixed manner, i.e. non-releasably.

In another preferred illustrative embodiment of the present invention, provision can be made that the micro-stimulating elements are actuators, which generate a vibration, a tactile impulse or jolt, or are means for generating local and/or short heat or cold impulses, of which the intensity and/or temperatures are continuously modifiable. It is thus conceivable that the rest generates a sensory impulse or a sensation whose intensity can be increased or reduced until it is perceived by the person or is no longer perceived. By way of the control device, this variability can be actively or passively regulated by the person being treated. Thus, the rest according to the invention also proves suitable for the outpatient sector or for use at home.

Moreover, according to the invention, provision can advantageously be made that the micro-stimulating elements constitute sources of electromagnetic radiation, in particular punctiform light sources, of which the wavelength and/or intensity are preferably variable. It has been shown that irradiation with light of a specific wavelength, in particular of several wavelengths, can have a particularly advantageous effect on the wellbeing of the human body. In addition to the warming effect of infrared radiation, irradiation with light of shorter wavelengths, for example from the visible solar spectrum, also gives a very pleasant feel. This irradiation can be set individually by each person, both in wavelength and also in intensity. This variation of the wavelength and of the intensity can be realized, for example, by means of filters and a white-light source.

Preferably, provision can also be made that the control device is connected wirelessly to the micro-stimulating elements, in particular that the control device is a mobile communication appliance with a corresponding application for actuation of the micro-stimulating elements and with a memory for storing data. For example, the control device can be a tablet or a smartphone on which a suitable app is installed. In this way, the data relating to the treatment or to the training can be transferred automatically to different appliances, such that the person carrying out the treatment is always kept informed of the progress of the treatment. It also proves particularly advantageous that the data are stored permanently and continuously. Thus, the data from a previous training session can be retrieved and can be compared with the current training data. The progress of the training can be evaluated through this comparison of the values, as a result of which the subsequent course of the training or treatment can be adapted individually to the person to be treated. The person can also be shown this training progress directly on the tablet or the smartphone, such that said person can himself evaluate the training progress. Therefore, the rest is suitable not only as a clinical therapy instrument, but instead also as a training appliance for private use.

In particular, provision can be made according to the invention that the micro-stimulating elements are dimensioned, in particular with a diameter of 1 to 10 mm, preferably of 2 to 5 mm, in such a way that, in the inactive state, they are not perceptible to the person on the rest. Thus, the rest can also be used as a conventional mattress support system. The dimensions of the micro-stimulating elements mean that they do not provide stimulation in the inactive state, since they are not perceptible to the person. It is only when the micro-stimulating elements are activated by the control device that the elements are perceptible by way of the stimuli that are produced.

A method for achieving the aforementioned object is a method for sensory training of a person using a rest for a mattress support system, in particular for a mattress of an item of furniture for sleeping on, sitting on or lying on, wherein the rest has a multiplicity of micro-stimulating elements which can be actuated individually or in groups and trigger sensory stimuli and which can be actively or passively controlled by the person, either individually or in groups, via a control device, wherein the passive control involves the use of a feedback signal which is generated by the control device on the basis of the reaction of the person to the stimuli. Accordingly, a method is provided for sensory training of a person using a rest for a mattress support system, in particular for a mattress of an item of furniture for sleeping on, sitting on or lying on, wherein the rest has a multiplicity of micro-stimulating elements which can be actuated individually or in groups and trigger sensory stimuli. These elements are actively or passively controlled by the person, either individually or in groups, via a control device. The passive control involves the use of a feedback signal which is generated by the control device on the basis of the reaction of the person to the stimuli. By generating this feedback signal, the person can directly influence the training. At the same time, a person carrying out the treatment obtains direct information concerning the state of training of the person or the training progress that the person has made. This method thus permits an improvement in the efficiency of the training and/or of the treatment.

In particular, according to the present invention, provision can moreover be made that the micro-stimulating elements are actuated variably in order to generate a stimulus pattern or to modify or stabilize a body image of the person, wherein in particular the micro-stimulating elements are activated in such a way that their intensity and the spacing from an adjacent active micro-stimulating element are varied according to the feedback signal of the person. Through this dependency of the actuation of the elements on the detected stimuli, it is possible to establish a particularly exact body image of the person. Furthermore, during the training, any change of this body image can be examined. The activation of the micro-stimulating elements can be changed depending on the development of the stimulus pattern and/or of the body image. In this way, both the person to be treated and the person carrying out the treatment have the possibility of controlling the training, and the training progress, actively and thus very efficiently.

Preferably, provision can also be made according to the invention that the micro-stimulating elements are actuated in such a way that the sensory stimulus produced is only just perceptible to the person and/or in such a way that it is possible only just to differentiate between two locally separate stimuli, wherein this stimulus threshold, in a subsequent training step, is actuated again by the control device, and the feedback signals of the person are compared. Through this test of the discrimination threshold of the person, it is possible to measure the body image of the person and to use this as a basis for more efficient training. This method has proven particularly helpful for the treatment of Parkinson's, dementia, decubitus ulcers, or coma patients.

Provision can also be made according to the present invention that the feedback signals of the person in response to the stimuli of the micro-stimulating elements are generated actively by the person or passively by sensors. Using the control unit, the person can actively and directly generate a feedback signal, by which the person expresses the fact that he has not felt the stimulus at all or that he has felt it clearly or poorly. Moreover, using the control device, the person is able to specify at which position the stimulus or the stimuli were perceived. In the case of a comatose person, for example, the stimuli can be demonstrated by sensors directly on the body of the person. These sensors in turn generate a feedback signal via the control device, which feedback signal serves in turn to adapt the subsequent treatment or the subsequent training individually to the person.

In another particularly advantageous illustrative embodiment of the present invention, provision can be made that, for targeted training purposes, micro-stimulating elements for selected body regions or body parts are activated in order to be able to transmit activating impulses to internal organs or in order to achieve a targeted change of the representation of one or more body parts in the neuronal system of the person. In this way, for almost any disease or condition, the training can be adapted individually to the person in order to achieve maximum efficiency of training.

Moreover, according to the invention, provision can preferably be made that the activating impulses are controlled such that metabolic processes or the blood circulation are modified in the stimulated regions or throughout the body. Through this modification of the blood circulation and/or of the metabolic process at specific points, traumatized regions of the body can be treated in a targeted manner. However, it is not only therapeutic treatments that can be carried out by the invention or by the method according to the invention, but also treatments that improve the general sense of wellbeing, i.e. wellness.

Moreover, provision can preferably be made that a rest for a mattress support system as claimed is used to carry out the method according to the invention.

### BRIEF DESCRIPTION OF THE DRAWINGS

A preferred illustrative embodiment of the present invention is explained in more detail below with reference to the drawing, in which:

FIG. 1 shows a perspective view of a rest with a person; and

FIG. 2 shows a view of the person shown in FIG. 1 on the rest.

### DETAILED DESCRIPTION OF PREFERRED EMBODIMENTS

The rest 10 according to the invention, shown here by way of an example, is a rest for a mattress support system of an item of furniture (not shown) for sleeping on or lying on. This rest 10 shown in the figures has the shape of a rectangular solid and is dimensioned in such a way that an adult person 13 of average height has space to lie on it. At the same time, it should be noted that the rest according to the invention may also be used for seat furniture or the like.

The rest 10 shown here serves primarily for sensory training of a person 13 who is to be treated. In addition, however, the rest 10 can also be used for massage purposes or other wellness purposes. For this training or this purpose, the rest 10 shown in the figures has on its surface 11 a multiplicity of micro-stimulating elements 12. These micro-stimulating elements 12 can be distributed in any desired manner across the entire surface 11 of the rest 10. It is equally conceivable that the micro-stimulating elements 12 are integrated in the rest 10 or are arranged on an underside of the rest 10.

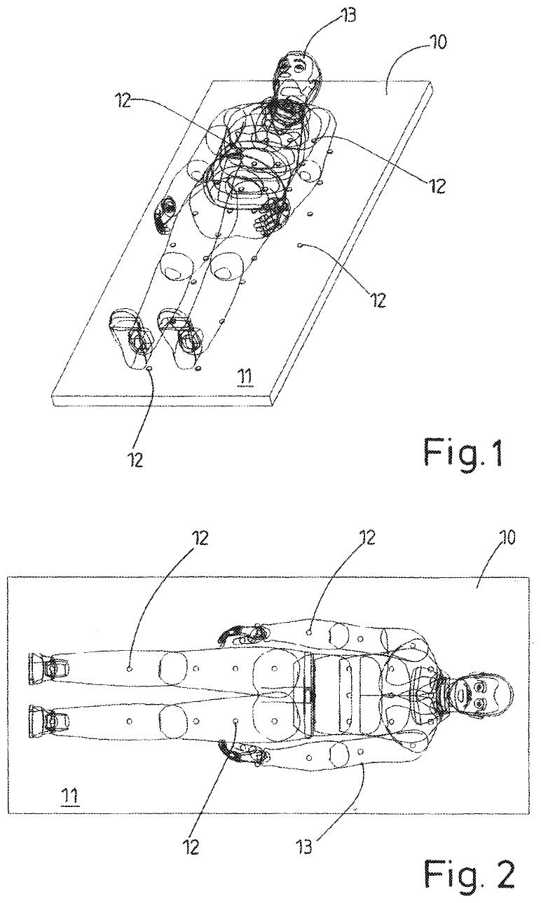

For the method according to the invention, the micro-stimulating elements are arranged on the surface 11 of the rest 10 in such a way that at least almost all the body regions of the person 13 to be treated can be reached. FIGS. 1 and 2 shows a schematic representation of a person 13 lying on the rest 10. It will be seen in particular from FIG. 2 that the micro-stimulating elements 12 are arranged under all of the limbs and the body of the person 13. That is to say, micro-stimulating elements 12 are arranged under the feet, under the legs and also under the arms and hands, etc.

To take account of different sizes and shapes of bodies, provision can be made that the micro-stimulating elements 12 can be secured releasably on the surface 11, for example by a hook-and-loop fastener. Alternatively, however, provision is also made that the entire surface 11 of the rest 10 is covered by a fine network of the micro-stimulating elements 12, such that all parts of the body can be reached by the micro-stimulating elements 12 independently of the body shape of the person 13. In addition, provision can be made that certain body parts, for example a thigh or the feet, are assigned fewer or more micro-stimulating elements 12 and that other body regions such as the back or the head are assigned more or fewer micro-stimulating elements 12. Moreover, the spacing between adjacent micro-stimulating elements 12 can vary depending on the body region of the person 13. For example, it is conceivable that the expected position of the back of the person 13 to be treated is assigned a higher density of micro-stimulating elements 12 per surface area than the regions of the lower leg.

The micro-stimulating elements 12 can be actuators which generate a vibration, a tactile impulse or a mechanical jolt, or they can be means for generating local and/or short heat or cold impulses, of which the intensities and temperatures are continuously variable. Moreover, the micro-stimulating elements 12 can be sources of electromagnetic radiation, for example light sources whose wavelength and/or intensity are variable. By means of these micro-stimulating elements 12 or a combination of them, the person 13 to be treated can be suitably treated according to the clinical picture and the nature of the treatment or use. For example, it is conceivable that heat is generated at points in the lumbar region, while the left leg of the person 13 is stimulated by periodic, mechanical impulses or current surges.

The micro-stimulating elements 12 can be powered wirelessly or by wire. Accordingly, it is conceivable for example that the micro-stimulating elements 12 have a battery. It is equally conceivable that the micro-stimulating elements 12 obtain their energy via electromagnetic radiation, or that the elements 12 are connected by wire to a corresponding energy source.

Depending on the nature of the elements 12 and their use, they can differ from each other in size. According to the invention, provision is made that the elements 12 have a diameter of 1 to 10 mm, preferably 2 to 5 mm. It is preferable according to the invention that the micro-stimulating elements 12 are dimensioned and arranged in such a way that, in the inactive state, they are not perceptible to the person 13.

For the sake of clarity, only some of the circular micro-stimulating elements 12 shown have been provided with a reference sign in FIG. 1 and FIG. 2.

For the control of the micro-stimulating elements 12, a control device (not shown) is assigned to the rest 10. This control device can communicate with all of the micro-stimulating elements 12 by wire or wirelessly. Individual micro-stimulating elements 12, groups of these micro-stimulating elements 12 or all of the elements 12 can be actuated or regulated by means of this control device. For particularly ergonomic handling of the control device, provision is made that the latter is designed for example as a tablet PC, smartphone or the like. Using this tablet PC, the person carrying out the treatment or the person 13 to be treated is able to control the micro-stimulating elements 12 preferably via an app. It is thus conceivable that programs specific to a person or programs specific to a clinical picture are stored on the control device, such that the person 13 is able independently to carry out the sensory training or the treatment. It is of great importance here that the control unit not only makes it possible to initiate and control the training but also allows the person 13 to input feedback. In this way, the person 13 to be treated can independently input information about the body position in which a stimulus is perceived. Depending on this perception, the person 13 can independently increase the intensity of the stimulus at certain points or overall or can reduce the intensity to below a certain perception threshold.

In addition, the person 13 can determine via the control device to what extent he can distinguish between micro-stimulating elements 12 spaced apart from one another. Through the actuation of several adjacent micro-stimulating elements 12, the spacing or the so-called discrimination threshold between two micro-stimulating elements 12 still to be differentiated from each other can be measured and improved. By virtue of the permanent and preferably continuous storage of the feedback signals of the person 13, previous training data can be retrieved and compared with current training data, in order thereby to be able to assess the training progress. Depending on this training progress, the subsequent treatment program can be tailored individually to the person 13.

By virtue of this targeted stimulation of the person 13 at certain points or overall, and by virtue of the feedback from said person 13 in response to the individual stimuli, a body image of the person 13 as a whole can be established, which body image allows the person carrying out the treatment to obtain information concerning the disease or the progress of the treatment.

In addition to the above-described active actuation of the micro-stimulating elements 12 by the person 13, these elements can also be activated passively by the person 13. This passive actuation is advantageous in particular for the treatment of coma patients. For this passive actuation, suitable sensors have to be placed on the person 13, i.e. sensors are able to demonstrate that the person 13 has experienced a stimulus caused by the micro-stimulating elements 12. Using the information determined by the sensors, further signals can in turn be generated for actuation of the micro-stimulating elements 12, such that even a person 13 in a coma can be treated or trained in a targeted manner by the elements 12 integrated in the rest 10.

The micro-stimulating elements 12 permit the stimulation of muscles and also of metabolic processes. In addition, circulation of blood can be promoted. Moreover, stimuli are generated that serve to activate selected regions of parts of the body and to produce activating impulses on internal organs. In this way, targeted changes of the representation of one or more body parts in the neuronal system of the person 13 can be produced. These changes allow a large number of conditions to be treated in a manner that is specific to the person and thus effective.

### LIST OF REFERENCE SIGNS

10 rest
11 surface
12 micro-stimulating elements
13 person

1. A rest (10) for a mattress support system, in particular for a mattress of an item of furniture for sleeping on, sitting on or lying on, for sensory training of a person (13), comprising:
a multiplicity of micro-stimulating elements (12) which can be actuated individually or in groups and trigger sensory stimuli and which are arranged on a surface (11) of the rest (10) or are integrated in the rest (10); and
a control device having means for active or passive actuation of the micro-stimulating elements (12), individually or in groups, by the person (13) during the training and for generating a feedback signal in response to the sensory stimuli.

2. The rest (10) as claimed in claim 1, wherein the micro-stimulating elements (12) are arranged on or in the rest (10) at a spacing equal to, in particular below, the discrimination threshold of the human body for the perception of stimuli, this spacing being variable for different body regions of the person (13).

3. The rest (10) as claimed in claim 1, wherein the micro-stimulating elements (12) are actuators, which generate a vibration, a tactile impulse or jolt, or are means for generating local and/or short heat or cold impulses, of which the intensities and/or temperatures are continuously modifiable.

4. The rest (10) as claimed in claim 1, wherein the micro-stimulating elements (12) constitute sources of electromagnetic radiation, in particular punctiform light sources, of which the wavelength and/or intensity are preferably variable.

5. The rest (10) as claimed in claim 1, wherein the micro-stimulating elements (12) are releasably connectable to the rest (10), in particular in that the micro-stimulating elements (12) can be assigned in any desired manner to the rest (10).

6. The rest (10) as claimed in claim 1, wherein the control device is connected wirelessly to the micro-stimulating elements (12), in particular in that the control device is a mobile communication appliance with a corresponding application for actuating the micro-stimulating elements (12) and with a memory for storing data.

7. The rest (10) as claimed in claim 1, wherein the micro-stimulating elements (12) are dimensioned, in such a way that, in the inactive state, they are not perceptible to the person (13) on the rest.

8. A method for sensory training of a person (13) using a rest for a mattress support system, in particular for a mattress of an item of furniture for sleeping on, sitting on or lying on, wherein the rest (10) has a multiplicity of micro-stimulating elements (12) which can be actuated individually or in groups and trigger sensory stimuli and which can be actively or passively controlled by the person (13), either individually or in groups, via a control device, comprising generating a feedback signal by the control device on the basis of the reaction of the person (13) to the stimuli, wherein the passive control involves the use of a the feedback signal which is generated by the control device on the basis of the reaction of the person (13) to the stimuli.

9. The method as claimed in claim 8, wherein the micro-stimulating elements (12) are actuated variably in order to generate a stimulus pattern or to modify or stabilize a body image of the person (13), wherein in particular the micro-stimulating elements (12) are activated in such a way that their intensity and the spacing from an adjacent active micro-stimulating element (12) are varied according to the feedback signal of the person (13).

10. The method as claimed in claim 8, wherein the micro-stimulating elements (12) are actuated in such a way that the sensory stimulus produced is only just perceptible to the person (13) and/or in such a way that it is possible only just to differentiate between two locally separate stimuli, wherein this stimulus threshold, in a subsequent training step, is again actuated by the control device, and the feedback signals of the person (13) are compared.

11. The method as claimed in claim 8, wherein the feedback signals of the person (13) in response to the stimuli of the micro-stimulating elements (12) are generated actively by the person (13) or passively by sensors.

12. The method as claimed in claim 8, wherein, for targeted training purposes, micro-stimulating elements (13) for selected body regions or body parts are activated in order to be able to transmit activating impulses to internal organs or in order to achieve a targeted change of the representation of one or more body parts in the neuronal system of the person (13).

13. The method as claimed in claim 8, wherein the activating impulses are controlled such that metabolic processes or the blood circulation are modified in the stimulated regions or throughout the body.

14. The method as claimed in claim 8, wherein a rest (10) for a mattress support system is used to carry out the method.

15. The method as claimed in claim 14, wherein the rest (10) comprises:
a multiplicity of micro-stimulating elements (12) which can be actuated individually or in groups and trigger sensory stimuli and which are arranged on a surface (11) of the rest (10) or are integrated in the rest (10); and
a control device having means for active or passive actuation of the micro-stimulating elements (12), individually or in groups, by the person (13) during the training and for generating a feedback signal in response to the sensory stimuli.

16. The rest (10) as claimed in claim 7, wherein the micro-stimulating elements (12) are dimensioned with a diameter of 1 to 10 mm.

17. The rest (10) as claimed in claim 7, wherein the micro-stimulating elements (12) are dimensioned with a diameter of 2 to 5 mm.
